(19) United States
(12) Patent Application Publication
(10) Pub. No.: US 2026/0232423 A1
(43) Pub. Date: Aug. 13, 2026

(54) APPARATUS AND METHOD FOR ANIMAL BLOOD COLLECTION

(71) Applicant: UTI Limited Partnership, Calgary (CA)

(72) Inventors: Mathieu René Robert PRUVOT, Cochrane (CA); Thomas Michael LIJNSE; Colin DALTON, Calgary (CA); Syed Kazim HAIDER, Calgary (CA)

(21) Appl. No.: 19/159,249

(22) PCT Filed: Feb. 26, 2024
(86) PCT No.: PCT/CA2024/050229

§ 371 Date: Aug. 22, 2025

Related U.S. Application Data
(60) Provisional application No. 63/486,773, filed on Feb. 24, 2023.

(51) Int. Cl. A61D 99/00; F42B 12/54

(52) U.S. Cl. CPC A61D 99/00; F42B 12/54

(57) ABSTRACT

Apparatuses and methods are disclosed for gathering biological samples from medium to large terrestrial mammals, such as gathering a blood sample from an animal. A remote blood collection apparatus, or dart, includes a fluid collection reservoir maintaining a vacuum, and one or more sharpened tubular ends, which may be one or more needles. In response to the apparatus being driven into an animal, a continuous passage is created from the fluid collection reservoir through the one or more sharpened tubular ends. The apparatus is configured to extract, using the vacuum in the fluid collection reservoir and from the animal, fluid into the fluid collection reservoir from the animal. The apparatus provides advantages over known blood collection techniques, as well as known biopsy needles that take a sample of core flesh along with blood and may result in inaccurate sample analysis.

### CROSS-REFERENCE TO RELATED APPLICATIONS

This application claims priority to U.S. Provisional Patent Application number 63/486,773 filed Feb. 24, 2023, which is incorporated by reference in its entirety.

### FIELD

The present disclosure relates to blood collection, including but not limited to animal or wildlife blood collection.

### BACKGROUND

Wildlife disease surveillance is a key component of understanding the risk of zoonotic disease emergence and tracking the effect of ecological changes on wildlife health. Although the testing for pathogens and other biomarkers increasingly uses non-invasive sampling methods (e.g. fecal samples), the types of tests that can be done on such samples are limited and not always optimal. Collection of blood for laboratory testing remains a gold standard for most biomarkers of health and disease.

Current methods of blood collection in wildlife require capture, restraint and/or anesthesia, which is stressful to the animals, can result in injuries for both animal and humans involved, and requires costly equipment such as a helicopter. A growing number of biological markers related to stress (e.g. cortisol/corticosterone), immune function (e.g. Leucocyte coping capacity, Bacterial Killing Assay) and oxidative stress (e.g. Reactive Oxygen Species) provide important indication on the effect of environmental changes or anthropogenic disturbances on wildlife. However, the high stress involved in current blood collection techniques interferes with the measurement of these biomarkers and challenge the ability to make reliable inference on the effect of environmental changes on wildlife physiology.

Improvements is approaches for animal blood collection are desirable.

### BRIEF DESCRIPTION OF THE DRAWINGS

Embodiments of the present disclosure will now be described, by way of example only, with reference to the attached Figures.

FIG. 1 illustrates an apparatus for remote blood collection, in accordance with one or more embodiments.

FIG. 2 illustrates an apparatus for remote blood collection including multiple bore needles, in accordance with one or more embodiments.

FIG. 3 illustrates an apparatus for remote blood collection with a mechanism adapted to hold several small needles, in accordance with one or more embodiments.

FIG. 4 illustrates an apparatus for remote blood collection using a large central needle, in accordance with one or more embodiments.

FIG. 5 illustrates an example of a Luer-lock tipped tranquilizer dart, in accordance with one or more embodiments.

### DETAILED DESCRIPTION

Apparatuses and methods are disclosed for gathering biological samples from medium to large terrestrial mammals, such as gathering a blood sample from an animal. A remote blood collection apparatus, or dart, includes a fluid collection reservoir maintaining a vacuum, and one or more sharpened tubular ends, which may be one or more needles. In response to the apparatus being driven into an animal, a continuous passage is created from the fluid collection reservoir through the one or more sharpened tubular ends. The apparatus is configured to extract, using the vacuum in the fluid collection reservoir and from the animal, fluid into the fluid collection reservoir from the animal. The apparatus provides advantages over known blood collection techniques, as well as known biopsy needles that take a sample of core flesh along with blood and may result in inaccurate sample analysis.

For the purpose of promoting an understanding of the principles of the disclosure, reference will now be made to the features illustrated in the drawings and specific language will be used to describe the same. It will nevertheless be understood that no limitation of the scope of the disclosure is thereby intended. Any alterations and further modifications, and any further applications of the principles of the disclosure as described herein are contemplated as would normally occur to one skilled in the art to which the disclosure relates. It will be apparent to those skilled in the relevant art that some features that are not relevant to the present disclosure may not be shown in the drawings for the sake of clarity.

Certain terms used in this application and their meaning as used in this context are set forth in the description below. To the extent a term used herein is not defined, it should be given the broadest definition persons in the pertinent art have given that term as reflected in at least one printed publication or issued patent. Further, the present processes are not limited by the usage of the terms shown below, as all equivalents, synonyms, new developments and terms or processes that serve the same or a similar purpose are considered to be within the scope of the present disclosure.

Embodiments of the present disclosure provide an adaptation to traditional tranquilizer darts to collect blood samples from wildlife by modifying existing darting systems to draw blood, instead of injecting a drug. Embodiments of the present disclosure provide a new design of an attachment head for a launched hypodermic dart to be used in remote blood collection, for example on large free-ranging wild animals and livestock. The dart body and dart tip work together.

The present disclosure addresses current hurdles in wildlife blood collection methods. Embodiments of the present disclosure introduce a novel design to adapt existing tranquilizer dart platforms in order to build a multifunctional dart platform (MDP) for comprehensive wildlife health monitoring. Embodiments of the present disclosure overcome the issues caused by standard or known sedation and tissue collection protocols by creating a remote extraction technique, for example based on the principles of small needles in a dart adaptor delivered using existing darting methods. Embodiments of the present disclosure may be provided as part of darting approaches including one or more of a pneumatic rifle, blowgun, or bow.

Some known biopsy darts take a core sample of flesh from the animal, which causes a lot of damage, taking a large section of tissue. These known approaches have no vacuum, which is more dangerous to the animal. The known biopsy dart is in the animal for a while, falls out, and typically takes a large chunk of flesh, which may contaminate and otherwise adversely affect blood sample readings. The longer the known biopsy dart is in the animal, the more the blood may have undesirable markers. In contrast, embodiments of the present disclosure provide a remotely fired version of a blood draw in a lab, such as for a blood test.

According to an embodiment, the present disclosure provides an apparatus for remote blood collection, in accordance with one or more embodiments. The apparatus may comprise a fluid collection reservoir configured to maintain a vacuum. The apparatus may comprise a tip in communication with the fluid collection reservoir. The apparatus may comprise one or more sharpened tubular end portions extending from the tip and configured to pierce an animal skin surface to enable fluid collection. The tip may be configured to, in response to the tip being driven into the animal, create a continuous passage from the fluid collection reservoir through the one or more sharpened tubular end portions into the animal, such that the vacuum in the fluid collection reservoir extracts fluid into the fluid collection reservoir from the animal.

In an example embodiment, the fluid collection reservoir comprises a sealed evacuated tube to independently maintain the vacuum by means of the seal. In an example embodiment, the sealed evacuated container for fluid collection may be a vacutainer, or similar container, such as those used for phlebotomy. In another example embodiment, the tip and the fluid collection reservoir cooperate to maintain the vacuum in the fluid collection reservoir.

In an example embodiment, the apparatus comprises a dart.

In an example embodiment, the fluid comprises capillary blood and the one or more sharpened tubular end portions are configured to enable collection of the capillary blood.

In an example embodiment, the one or more sharpened tubular end portions comprise one or more needles.

In an example embodiment, the one or more needles comprise at least one microneedle. In an example embodiment, the one or more needles comprise a plurality of needles of different heights, diameters, or other different physical characteristics.

In an example embodiment, the one or more needles comprise a solid needle configured for mechanical stability. In an example embodiment, the one or more needles comprise a hollow needle configured for fluid extraction. In an example embodiment, the one or more needles comprise a closed tip. In an example embodiment, the one or more needles comprise an open tip.

In an example embodiment, some of the one or more needles comprise one or more perforations on the needle surface perpendicular to the long axis of the needle. In an example embodiment, the one or more perforations may have equal or different sizes.

In an example embodiment, the one or more sharpened tubular end portions are configured to be actuated into the animal skin surface after initial contact. In an example embodiment, the one or more sharpened tubular end portions are configured to be retracted out of the animal skin surface after initial contact.

In an example embodiment, the apparatus is configured to perforate the animal skin surface and to extract fluids pooling on the animal skin surface by the vacuum.

In an example embodiment, the apparatus comprises a dart configured to be accelerated towards the target animal via a firing system. In an example embodiment, the firing system comprises a pneumatic rifle, a blowgun, a bow, or a spring loaded mechanism. In another example embodiment, the firing system comprises a manual firing system such as a stick or spear.

In an example embodiment, the apparatus further comprises a shock absorbing mechanism. The shock absorbing mechanism may comprise a shock absorbing cap that absorbs the majority of the force from the firing system upon being launched towards the animal.

In an example embodiment, the apparatus is configured to optimize aerodynamics by reducing the overall drag coefficient, which may include, but is not limited to, components of the apparatus having a streamlined shape, and/or the apparatus comprising additional components such as nose cones and stabilizer fins.

In another aspect, the present disclosure provides a method of remote animal blood collection comprising: creating a vacuum in a remote blood collection apparatus including a fluid collection reservoir and one or more sharpened tubular ends; in response to the apparatus being driven into an animal, creating a continuous passage from the fluid collection reservoir through the one or more sharpened tubular ends; and extracting, using the vacuum in the fluid collection reservoir and from the animal, fluid into the fluid collection reservoir from the target animal.

In an example embodiment, the method further comprises accelerating the remote blood collection apparatus towards the animal via a firing system.

In an example embodiment, extracting the fluid comprises extracting blood that is free of flesh of the animal.

In an example embodiment, the apparatus comprises a dart composed of two independent components that move relative to one another.

In an example embodiment, the apparatus further comprises a location indicator configured to enable location of the apparatus after fluid extraction. The location indicator may comprise a visual indicator, auditory indicator, wireless electronic indicator, or any combination thereof. The location indicator may be configured to operate in accordance with a wireless signal-based locating technology such as GPS (Global Positioning System), RFID (Radio Frequency Identifier), Bluetooth™, visual indicators such as bright colours or markings, or audible indicators.

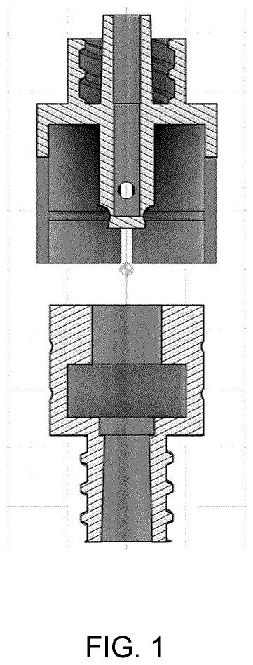

FIG. 1 illustrates a side view of an interlocking syringe head mechanism in accordance with one or more embodiments, configured to maintain a pressure difference until significant impact in the needle axial direction. The system may be constructed to be compatible with the universal Luer-lock mechanism.

In an example embodiment, the tip further comprises: at least two interlocking segments configured to enable the vacuum to be maintained in the fluid collection reservoir, the at least two interlocking segments configured to move, in response to the tip being driven into the animal, to create the continuous passage from the fluid collection reservoir through the one or more sharpened tubular end portions into the animal.

In an example embodiment, the at least two interlocking segments comprise a needle tip with sliding sheath configured to a hole on a side of a body of the one or more sharpened tubular end portions.

The example embodiment of FIG. 1 illustrates a two-part interlocking dart tip adaptor for use in gathering a blood sample from an animal. The dart tip may comprise one or more needles used for penetrating the skin of an animal at the proximal end. In an example embodiment, at least one of the one or more needles may have either a closed or open tip. In an example embodiment, at least one of the one or more needles may define one or more perforations. The perforations may be are made on the needle surface perpendicular to the long axis of the needle. The perforations may serve as inlets for fluid extraction and may reduce the likelihood of needle blockage upon piercing the animal's hide. The one or more perforations may have equal or different sizes.

A dart base may comprise an adapter for fastening to a chamber portion for receiving and holding blood. The chamber portion may comprise a proximal portion adapted to be removably fastened to the dart base.

The two parts of the adaptor may be configured to lock together, for example under a pressure fit or other method, to form an airtight seal that enables a vacuum to be maintained in the chamber. In an example embodiment, the vacuum may be enabled using a friction lock and an o-ring, such that when device impacts the animal, the impact opens up the vacuum. The vacuum in the body may be controlled by the tip, and the body maintains vacuum pressure. The tip may be a connecting system which comprises a blocked system when it is unused, so that nothing can pass through, and that after impact, things can pass through in either direction.

The dart tip adaptor may be configured such that, upon penetrating animal skin, a pathway to the chamber is opened through the needle(s) and the vacuum within the chamber pulls blood from the animal through the tip and into the chamber. The needle(s) may be driven into the animal through an actuation mechanism upon initial contact, such as, but not limited to, a spring-loaded actuator. In an embodiment, the dart adaptor is configured to fit existing Luer-lock systems used readily throughout the veterinary medicine field. In another embodiment, the dart adaptor is provided as a custom fit locking and attachment system.

There has been success in designing microneedles which allow for pain free and easy blood collection in humans. These microneedles may be designed to interact with subdermal capillaries without impacting dermal nerve endings responsible for pain perception. An apparatus in accordance with one or more embodiments may comprise one or more microneedles.

In an example embodiment, the apparatus comprises a dart composed of two independent components that move relative to one another. The first component may have a central cylindrical portion with ridges on its surface and a single double ended needle that runs through its center such that there is a sharp needle protruding from both ends of the central portion. The second component comprises a hollow body matching the shape of the first component, for example, the second component is also cylindrical if the first component is cylindrical. The first component is meant to be situated inside the second component. The interior of the second component has grooves that match the ridges on the first component, such that the relative movement of the two components can be limited by the positioning of the grooves and ridges on the first and second components. The second component may have one or more needles protruding from one end, and a sealed evacuated container situated at its other end. When the apparatus is fired towards an animal, the impact on the animal causes one end of the needle on the first component to penetrate the animal's hide, and the force causes relative motion between the two components such that the seal on the evacuated container is pierced by the other end of the needle on the first component, causing the pressure of the vacuum within the evacuated container to extract fluids from the animal. Meanwhile, the one or more needles on the second component also insert into the animal upon impact and serve as a mechanical anchor to holding the apparatus in the animal's hide during fluid extraction. In an example embodiment, the sealed evacuated container for fluid collection may be a vacutainer, or similar container, such as those used for phlebotomy.

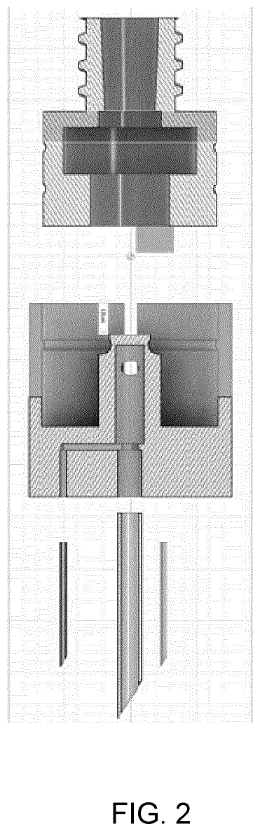

FIG. 2 illustrates an apparatus for remote blood collection including multiple bore needles, in accordance with one or more embodiments. The apparatus of FIG. 2 may be described as a modification of the embodiment of FIG. 1 to include multiple bore needles.

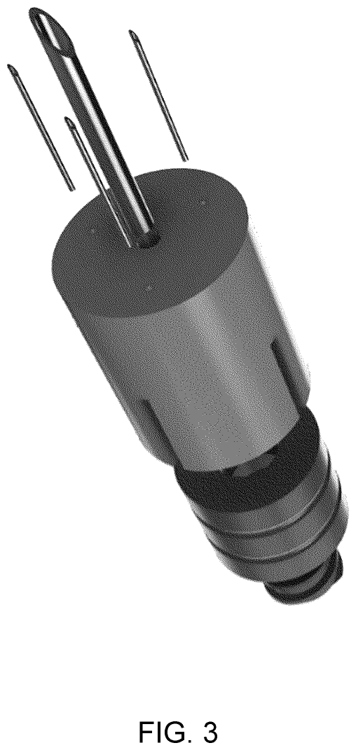

FIG. 3 illustrates an apparatus for remote blood collection with a mechanism adapted to hold several small needles, in accordance with one or more embodiments. The embodiment show in FIG. 3 illustrates further details and a perspective view of how the mechanism from FIG. 1 can be adapted to hold several smaller needles as in FIG. 2.

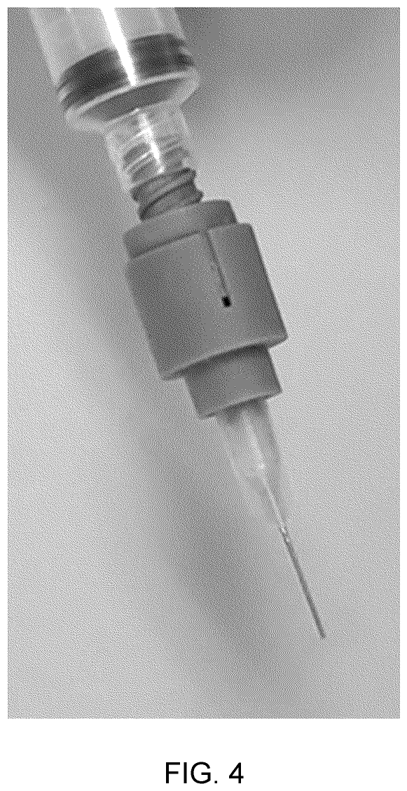

FIG. 4 illustrates an apparatus for remote blood collection using a large central needle, in accordance with one or more embodiments. The example shown in FIG. 4 is based on an apparatus produced by a stereolithographic (SLA) 3D printing. An apparatus in accordance with one or more embodiments may be produced using any number of fabrication methods, such as, but not limited to, injection molding, casting, computer numeric control (CNC) milling or turning.

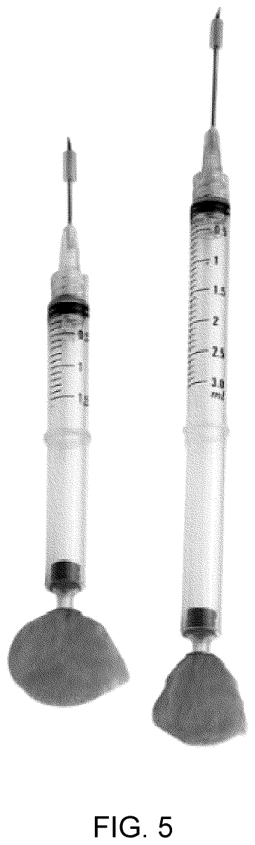

FIG. 5 illustrates an example of a Luer-lock tipped tranquilizer dart, in relation to which an apparatus in accordance with one or more embodiments of the present disclosure may be configured to adapt.

Embodiments of the present disclosure are configured to collect blood samples from wildlife by modifying an existing darting system to draw blood. Embodiments of the present disclosure may provide an improvement in wildlife health surveillance by facilitating access to appropriate samples for disease monitoring, and may improve the welfare of wild animals used in health monitoring studies by reducing the invasiveness of biological sampling. Embodiments of the present disclosure may be developed into a low-cost solution, which may facilitate large scale sampling across wildlife populations, beyond what is currently possible. The low-stress nature of the sampling procedure according to embodiments of the present disclosure may reduce interference in study of environmental changes on wild animal physiology and health, which is an important area of research at a time of unprecedented human-driven changes. In an example embodiment, an attachment head is provided for a launched hypodermic dart to be used in remote blood collection on large free-ranging wild animals and livestock.

In an example embodiment, the present disclosure provides a dart for use in gathering biological samples from medium to large terrestrial mammals. The dart may comprise: an adapter base for fastening to a fluid collection reservoir, for example a syringe; a dart tip comprising two or more interlocking segments that enable a vacuum to be maintained in the fluid collection reservoir, the dart tip having one or more sharpened tubular end portion(s) for piercing the skin and collecting capillary, venous, and/or arterial blood, for example one or more needles. When the dart tip is driven into the animal, interlocking sections may be configured to move to create a continuous passage from the fluid collection reservoir through the needle or needles into the animal. When a continuous connection is made, vacuum in the fluid collection reservoir may extract fluid into the reservoir chamber from the target animal.

In an example embodiment, the one or more needles may be of different heights, diameters, and other physical characteristics. The one or more needles comprise a plurality of needles of different heights, diameters, or other different physical characteristics such as open or closed tips, or perforations along the side of the needles.

In an example embodiment, the one or more needles may be solid for mechanical stability or hollow for fluid extraction.

In an example embodiment, the one or more needles may be mechanically or otherwise actuated into the skin after initial contact.

In an example embodiment, the one or more needles may be mechanically or otherwise retracted out of the skin after initial contact.

In an example embodiment, the fluid collected may comprise capillary blood from an animal.

In an example embodiment, the interlocking components may comprise a needle tip with sliding sheath that is configured to cover a hole on the side of the needle body.

In an example embodiment, the surface of the skin is perforated and fluids pooling on the surface may be extracted by the vacuum system.

In an example embodiment, the dart and dart tip may be accelerated towards the target animal through use of a firing system, such as a pneumatic rifle or blowgun.

In the preceding description, for purposes of explanation, numerous details are set forth in order to provide a thorough understanding of the embodiments. However, it will be apparent to one skilled in the art that these specific details are not required. In other instances, well-known mechanical structures are shown with limited details in order not to obscure the understanding.

The above-described embodiments are intended to be examples only. Alterations, modifications and variations can be effected to the particular embodiments by those of skill in the art without departing from the scope, which is defined solely by the claims appended hereto.

1. An apparatus for remote animal blood collection comprising:
a fluid collection reservoir configured to maintain a vacuum;
a tip in communication with the fluid collection reservoir; and
one or more sharpened tubular end portions extending from the tip and configured to pierce an animal skin surface to enable fluid collection,
the tip being configured to, in response to the tip being driven into the animal, create a continuous passage from the fluid collection reservoir through the one or more sharpened tubular end portions into the animal, such that the vacuum in the fluid collection reservoir extracts fluid into the fluid collection reservoir from the animal.

2. The apparatus of claim 1, wherein the fluid collection reservoir comprises a sealed evacuated tube to independently maintain the vacuum.

3. The apparatus of claim 1, wherein the tip and the fluid collection reservoir cooperate to maintain the vacuum in the fluid collection reservoir.

4. The apparatus of claim 1, wherein the tip further comprises:
at least two interlocking segments configured to enable the vacuum to be maintained in the fluid collection reservoir,
the at least two interlocking segments configured to move, in response to the tip being driven into the animal, to create the continuous passage from the fluid collection reservoir through the one or more sharpened tubular end portions into the animal.

5. The apparatus of claim 1, wherein the apparatus comprises a dart.

6. The apparatus of claim 1, wherein the fluid comprises capillary blood and the one or more sharpened tubular end portions are configured to enable collection of the capillary blood.

7. The apparatus of claim 1, wherein the one or more sharpened tubular end portions comprise one or more needles.

8. The apparatus of claim 7, wherein the one or more needles comprise at least one microneedle.

9. The apparatus of claim 7, wherein the one or more needles comprise a plurality of needles of different heights, diameters, or other different physical characteristics.

10. The apparatus of claim 7, wherein at least one of the one or more needles comprises an open tip.

11. The apparatus of claim 7, wherein the one or more needles comprises a closed tip.

12. The apparatus of claim 7, wherein at least one of the one or more needles comprises perforations along the side of the needle.

13. The apparatus of claim 7, wherein the one or more needles comprise a solid needle configured for mechanical stability.

14. The apparatus of claim 7, wherein the one or more needles comprise a hollow needle configured for fluid extraction.

15. The apparatus of claim 1, wherein the one or more sharpened tubular end portions are configured to be actuated into the animal skin surface after initial contact.

16. The apparatus of claim 1, wherein the one or more sharpened tubular end portions are configured to be retracted out of the animal skin surface after initial contact.

17. The apparatus of claim 1, further comprising a location indicator configured to enable location of the apparatus after fluid extraction.

18. The apparatus of claim 17 wherein the location indicator comprises a visual indicator, auditory indicator, wireless electronic indicator, or any combination thereof.

19. The apparatus of claim 4, wherein the at least two interlocking segments comprise a needle tip with sliding sheath configured to a hole on a side of a body of the one or more sharpened tubular end portions.

20. The apparatus of claim 1, wherein the apparatus is configured to perforate the animal skin surface and to extract fluids pooling on the animal skin surface by the vacuum.

21. The apparatus of claim 1, wherein the apparatus comprises a dart configured to be accelerated towards the target animal via a firing system.

22. The apparatus of claim 21, wherein the firing system comprises a pneumatic rifle, a blowgun, a bow, or a spring loaded mechanism.

23. A method of remote animal blood collection comprising:
creating a vacuum in a remote blood collection apparatus including a fluid collection reservoir and one or more sharpened tubular ends;
in response to the apparatus being driven into an animal, creating a continuous passage from the fluid collection reservoir through the one or more sharpened tubular ends; and
extracting, using the vacuum in the fluid collection reservoir and from the animal, fluid into the fluid collection reservoir from the target animal.

24. The method of claim 23 further comprising:
accelerating the remote blood collection apparatus towards the animal via a firing system.
